(19) United States
(12) Patent Application Publication
(10) Pub. No.: US 2016/0113931 A1
(43) Pub. Date: Apr. 28, 2016

(54) Method of treating and/or preventing neurodegenerative diseases

(71) Applicant: University of Macau, Macau (CN)

(72) Inventors: Ming Yuen LEE, Macau (CN); Ting Jun HOU, Suzhou (CN); Hui Dong YU, Guangzhou Science Town (CN)

(73) Assignee: University of Macau

(21) Appl. No.: 14/920,903

(22) Filed: Oct. 23, 2015

Related U.S. Application Data
(60) Provisional application No. 62/067,996, filed on Oct. 24, 2014.

(51) Int. Cl. A61K 31/519

(52) U.S. Cl. CPC A61K 31/519

(57) ABSTRACT

The present disclosure provides a method of treating and/or preventing neurodegenerative diseases, such as Parkinson disease, Alzheimer disease, Huntington's disease, multiple sclerosis and amyotrophic lateral sclerosis, comprising administrating a therapeutically effective amount of a compound of formula I or a pharmaceutically acceptable salt thereof to a subject in need thereof.

### CROSS-REFERENCE TO RELATED APPLICATION

This application claims benefit under 35 U.S.C. §119(e) of U.S. Provisional Application having Ser. No. 62/067,996 filed on 24 Oct. 2014, which is hereby incorporated by reference herein in its entirety.

### FIELD OF INVENTION

This disclosure relates to a method of treating and/or preventing neurodegenerative diseases.

### BACKGROUND OF INVENTION

Neurodegenerative diseases such as Parkinson disease (PD), Alzheimer disease (AD), Huntington's disease (HD), multiple sclerosis (MS) and amyotrophic lateral sclerosis (ALS) are resulted from chronic and progressive degeneration of neuronal populations in central nervous system (CNS). The incidence of these CNS disorders increases with age, becoming a global health concern and lead to the quality loss of elder life.

Recent studies indicate that stem/progenitor cells are able to secret numerous active factors (complement, cytokines, chemokines, trophic factors) which can modify the local microenvironment, contributing to neurorestorative processes. Oligodendrocytes, the myelin-forming glial cells of the CNS, not only provide myelination to long axons which enables rapid impulse propagation, but also serve to support neurons by producing neurotrophic factors and axon-glia metabolic coupling. Moreover, oligodendrocytes play a pivotal role in neurodegenerative diseases such as MS and AD. Oligodendroglial progenitor cells (OPCs) in adult CNS maintain the ability of regenerating oligodendrocytes that form new myelin sheaths following demyelinating injuries. OPCs also are considered a kind of stem cells may provide neuron regeneration and secret some neuroprotective proteins such as BDNF, NGF and NTF-3 to induce intracellular signals to prevent neurons from cell death and axonal degeneration.

### SUMMARY OF INVENTION

One example embodiment is a method of treating and/or preventing neurodegenerative disease. The method contains administrating a therapeutically effective amount of a compound of formula I or a pharmaceutically acceptable salt thereof to a subject in need thereof,

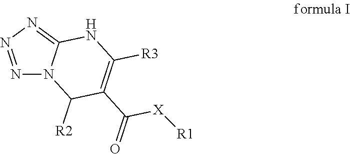

wherein R1, R2 and R3 are independently selected from the group consisting of H, unsubstituted or substituted (C1-C6) alkyl, (C1-C4) alkoxy, (C6-C12) aryloxy, and unsubstituted or substituted aryl; and X is C, O or N.

In another example embodiment, the neurodegenerative disease is selected from the group consisting of Parkinson disease, Alzheimer disease, Huntington's disease, multiple sclerosis and amyotrophic lateral sclerosis.

Another example embodiment is a pharmaceutical composition for treating and/or preventing neurodegenerative disease. The pharmaceutical composition contains a compound of formula I as described above and a pharmaceutically acceptable carrier.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1 shows the chemical structure of the compound (BHDPC) benzyl 7-(4-hydroxy-3-methoxyphenyl)-5-methyl-4,7-dihydrotetrazolo[1,5-a]pyrimidine-6-carboxylate.

FIGS. 2A and 2B show the chemical structures of compounds A-I.

FIG. 3A shows the schematic diagram for synthesizing compounds of formula I. FIG. 3B shows the schematic diagram for synthesizing the compound BHDPC.

FIG. 4 shows the cell viability of neuroblastoma SH-SY5Y cells upon treatment with BHDPC. Cells were treated with BHDPC (3 to 300 μM) for 24 h and the cell viability was measured by the MTT assay.

FIG. 5A shows the neuroprotective effect of BHDPC against MPP⁺-induced cytotoxicity in neuroblastoma SH-SY5Y cells. Cells were pre-treated with BHDPC (3, 10 and 30 μM) for 2 h and then incubated with or without 1 mM MPP⁺ for further 24 h. Cell viability was measured by the MTT assay. FIG. 5B shows the LDH assay of BHDPC against MPP+-induced cytotoxicity in SH-SY5Y cells. Cells were pre-treated with BHDPC (3, 10 and 30 μM) for 2 h and then incubated with or without 2 mM MPP⁺ for further 36 h. ###P<0.005 versus control group; **P<0.001, ***P<0.005 versus the MPP⁺-treated group was considered significantly different.

FIGS. 6A and 6B show that BHDPC attenuated MPP⁺-induced mitochondrial membrane potential (Δψm) loss and caspase 3 activity increase. After pre-treatment with 30 μM BHDPC or 0.1% DMSO (vehicle control) for 2 h, SH-SY5Y cells were incubated with or without 2 mM MPP⁺ for another 36 h. (FIG. 6A) Δψm was determined by the JC-1 assay. (FIG. 6B) Quantification of caspase 3 activity was determined by the Caspase 3 activity assay. ###P<0.005 versus control group; ***P<0.005 versus MPP⁺-treated group was considered significantly different.

FIGS. 7A and 7B shows BHDPC activated PKA signaling pathway. Cells were pre-treated with 30 μM BHDPC for 2 h. The cells were collected at 0, 30, 60, 90, and 120 mM FIG. 7A shows the western blot analysis of phosphorylated PKA upon BHDPC treatment. FIG. 7B shows the quantitation analysis of the expression of phosphorylation PKA upon BHDPC treatment. ***P<0.005 versus 0 h group was considered significantly different.

FIGS. 8A, 8B, 8C and 8D shows BHDPC activated PKA/CREB/Bcl2 signaling pathway. Cells were pre-treated with BHDPC (3, 10 and 30 μM) for 2 h and then were collected at 120 min. FIG. 8A shows the western blot analysis of phosphorylated PKA, phosphorylated CREB and Bcl2. The expression ratios of phosphorylated PKA, phosphorylated CREB and Bcl2 proteins as detected by Western blotting with specific antibodies were shown in FIGS. 8B, 8C and 8D respectively.

FIG. 9 shows the effect of PKA inhibitor, H89, on BHDPC's neuroprotection.

FIGS. 10A, 10B and 10C show the effects of BHDPC in oligodendroglial progenitor cells (OPCs) proliferation. OPCs were incubated with the drugs at indicated concentrations in proliferation medium for 2 days. (FIG. 10A) The cell viability was measured by MTT assay. The proliferative activity was measured by EdU assay (FIG. 10B) or immunostained with anti-Ki67 (the biomarker of cell proliferation) (FIG. 10C) with anti-oligo2 (the biomarker of oligodendrocyte lineage cells). **p<0.001, ***p<0.005 versus control group was considered significantly different.

FIGS. 11A and 11B shows that evaluation of pro-survival effects of BHDPC in OPCs. OPCs were incubated with 30 μM BHDPC or 0.1% DMSO (vehicle control) for 2 h followed by the addition or not of 200 μM H₂O₂ for a further 6 h (FIG. 11A) or LPS (FIG. 11B) for 24 h. Cell viability was measured by the MTT assay ###P<0.005 versus control group; ***p<0.005 versus control group was considered significantly different.

FIGS. 12A and 12B show that BHDPC prevented MPP⁺-induced neuronal death in cerebellar granule neurons (CGN) and cerebellar slice culture, respectively. Cerebellar tissue slice were treated with 30 μM BHDPC with or without MPP⁺ for 72 h and then were stained with PI. The slices were fixed and stained with anti-NeuN antibody and Hoechst dye. ###P<0.005 versus control group; **P<0.01, ***P<0.005 versus MPP⁺-treated group was considered significantly different.

FIG. 13 shows BHDPC prevented MPP⁺-induced neuronal death in primary cortical neurons. *P<0.01 versus control group, **P<0.01 versus MPP⁺-treated group was considered significantly different.

FIG. 14 shows the effects of BHDPC on α-tubulin and MAP2 expressions in primary cortical neurons for 14 days. Primary culture of cortical neurons was prepared from embryonic day 18±0.5 Sprague-Dawley rats. One day after seeding, primary cortical neurons were treated with 10 and 30 μM BHDPC for 14 days. Fourteen days following BHDPC treatment, primary cortical neurons cultured on glass coverslips (Thermo Scientific) were fixed with 4% paraformaldehyde for 15 min. Following fixation, neurons were permeabilized with 0.1% Triton X-100 in Tris-buffered saline for 7 min, and blocked with 4% bovine serum albumin and 2% goat serum for 1 h. α-tubulin and MAP2 were incubated at 1:400 dilution for 1 h at room temperature and overnight at 4° C. respectively. Following primary antibody incubation, neurons were incubated with Alexa Flour 488 or 568 secondary antibodies (anti-rabbit or anti-mouse) at 1:400 dilution for 1 h at room temperature. The coverslips were then mounted on microscopic slides (Thermo Scientific) using ProLong® Gold Antifade Mountant (Molecular Probes). Neurons were imaged using the LSM 780 laser scanning microscope (Carl Zeiss). Fluorescence intensity was measured using Image J.

FIG. 15A shows the effect of BHDPC on MPTP-induced dopaminergic neuron loss in zebrafish. Zebrafish at 1 dpf were exposed to BHDPC (3, 10, 30 μM) with or without MPTP for 48 h. Then fish were fixed for whole mount immunostaining. The morphology change of dopaminergic neurons in zebrafish brain was indicated by immunostaining with antibody against tyrosine hydroxylase (TH). Statistical analysis of TH density was accessed in each 10 fish/group. Data are expressed as a percentage of the control group. ###p<0.005, ***p<0.005 versus MPTP group was considered significantly different. FIG. 15B shows BHDPC protected against MPTP-induced DA neuron loss in zebrafish. Zebrafish at 1 dpf were exposed to different concentrations of BHDPC with or without MPTP for 48 h. Then fish were fixed for whole mount immunostaining.

FIG. 16 shows BHDPC attenuated the deficit of locomotion behavior on zebrafish larval induced by MPTP. Three dpf zebrafish embryos were co-incubated with 10 μM MPTP and BHDPC at the indicated concentrations for 96 hours, and zebrafish larval co-treated with MPTP and 150 μM L-dopa was used as positive control. After treatment, zebrafish were collected to perform locomotion behavior test using Viewpoint Zebrabox system and total distances travelled in 10 min were calculated.

FIG. 17 shows a stick model of BHDPC docked into the ATP-binding site of ROCK1.

### DETAILED DESCRIPTION OF THE PREFERRED EMBODIMENTS

As used herein and in the claims, “comprising” means including the following elements but not excluding others.

The inventors have identified the compounds of formula I as described below as a novel agent for treating and/or preventing neurodegenerative diseases.

### Example 1

### Compounds, Materials and Methods

1.1. Compounds:

The disclosure is directed to compounds of formula I:

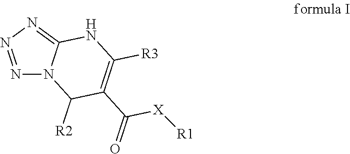

wherein R1, R2 and R3 are independently selected from the group consisting of H, unsubstituted or substituted (C1-C6) alkyl, (C1-C4) alkoxy, (C6-C12) aryloxy, and unsubstituted or substituted aryl; and X is C, O or N, or a pharmaceutically acceptable salt thereof.

In an embodiment, R1, R1, R2 and R3 are independently selected from the group consisting of H, unsubstituted or substituted (C1-C6) alkyl, (C1-C4) alkoxy, (C6-C12) aryloxy, and unsubstituted or substituted (C6-C12) aryl.

In an embodiment, R3 is halo or halo substituted straight or branched (C1-C6) alkyl.

In an embodiment, R1 and R2 are independently selected from the group consisting of five or six-membered N-heterocycle and benzoheterocycle.

In another embodiment, R1, R2 and R3 are independently (C1-C4) alkyl.

In another embodiment, R1, R2 and R3 are independently selected from the group consisting of phenylmethoxyl, phenylethoxyl or phenylpropoxyl.

In another embodiment, R1 and R2 are independently selected from the group consisting of phenyl, chlorobenzene, phenol or aniline.

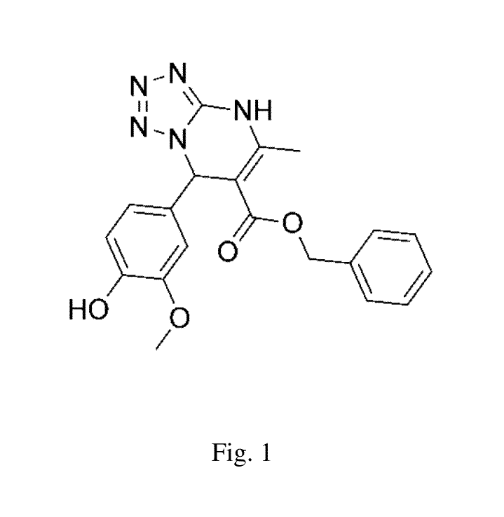

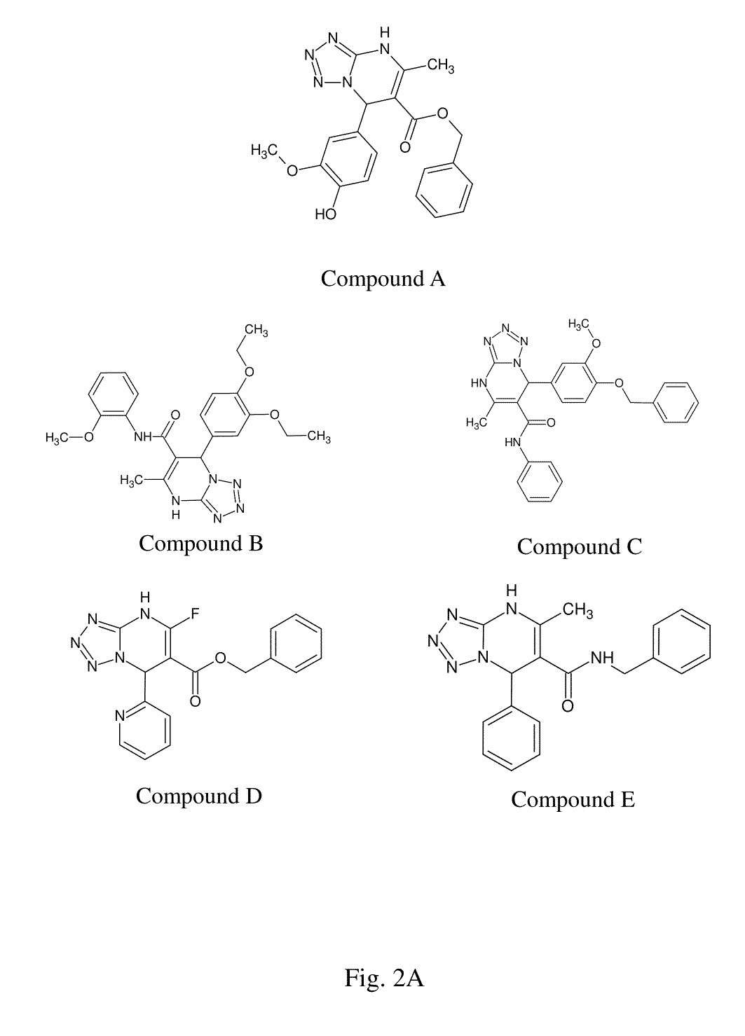

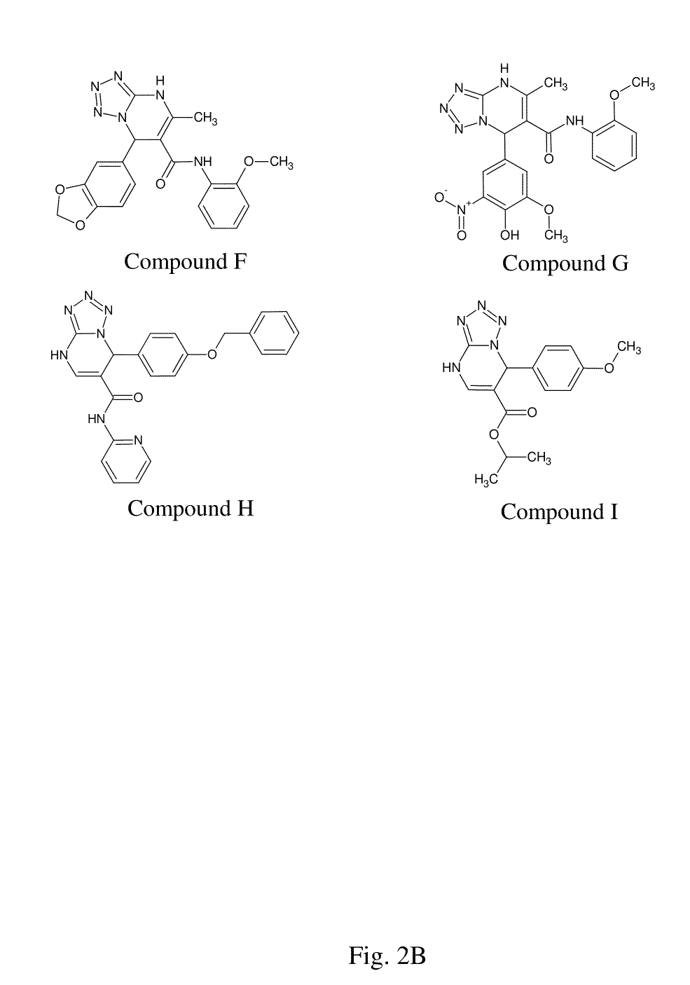

In another embodiment, the compound is selected from the group consisting of benzyl 7-(4-hydroxy-3-methoxyphenyl)-5-methyl-4,7-dihydrotetrazolo[1,5-a]pyrimidine-6-carboxylate (BHDPC), 7-(4-hydroxy-3-methoxylphenyl)-5-methyl-6-carboxylic acid benzyl ester-4,7-dihydrotetrazolo[1,5-α]pyrimidine (compound A); 7-(3,4-diethoxylphenyl)-6-[N-(2-methoxylphenyl)methanamide]-5-methyl-4,7-dihydrotetrazolo[1,5-α]pyrimidine (compound B); 7-(4-phenylmethoxyl-3-methoxylphenyl)-6-(N-phenyl methanamide)-5-methyl-4,7-dihydrotetrazolo[1,5-α]pyrimidine (compound C); 7-(2-N-pyrimidine)-5-fluor-6-carboxylic acid benzyl ester-4,7-dihydrotetrazolo[1,5-a]pyrimidine (compound D); 7-phenyl-6-(N-phenylmethyl)methanamide-5-methyl-4,7-dihydrotetrazolo[1,5-α]pyrimidine (compound E); 7-(1,3-benzodioxol-pentene)-6-[N-(2-methoxylphenyl)methanamide]-5-methyl-4,7-dihydrotetrazolo[1,5-α]pyrimidine (compound F); 7-(3-methoxy-3′-nitro-4-hydroxylphenyl)-6-[N-(2-methoxylphenyl)methanamide]-5-methyl-4,7-dihydrotetrazolo[1,5-α]pyrimidine ((compound G); 7-(4-phenylmethoxylphenyl)-6-[N-(2-pyridine)methanamide]-5-methyl-4,7-dihydrotetrazolo[1,5-α]pyrimidine (compound H); and 7-(4-methoxylphenyl)-6-carboxylic acid isopropyl ester-5-methyl-4,7-dihydrotetrazolo[1,5-α]pyrimidine (compound I). FIGS. 1, 2A and 2B show the chemical structures of BHPDC and compounds A-I respectively.

1.2. Synthesis of Compounds

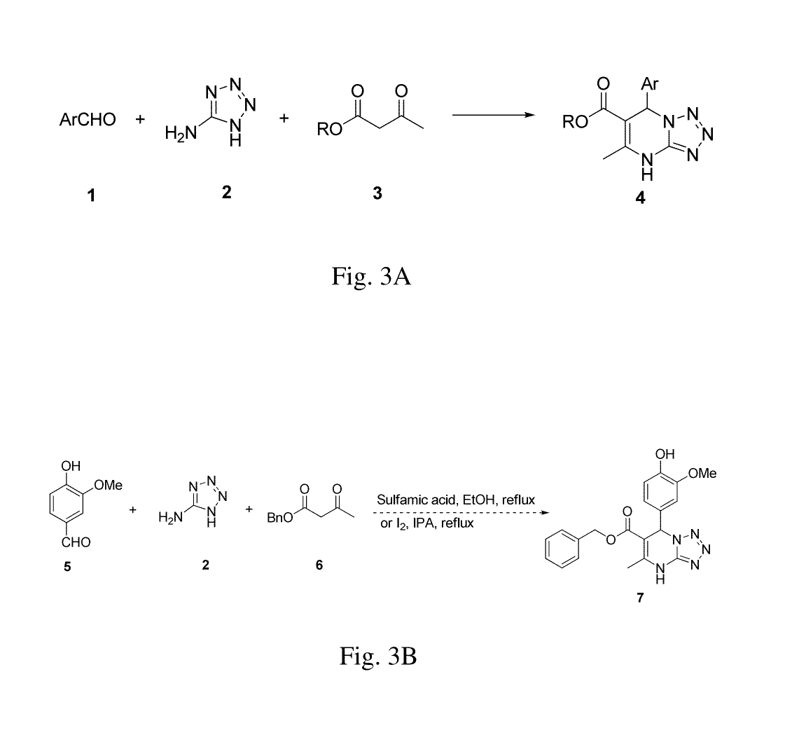

As shown in FIG. 3A, the core structure of BHDPC can be synthesized by reacting three compounds (1, 2, and 3). This reaction can be carried out at a solvent-free condition by increasing the temperature to 130-170° C. After addition of sulfamic acid as catalyst, the temperature of reaction may be reduced to 85° C. under a solvent-free or ethanol condition. Alternatively, a good yield can be obtained using 10% of iodine as catalyst and isopropanol as solvent under reflux condition.

As shown in FIG. 3B, compound 7 can be obtained by one-pot synthesis using the three commercially available reagents of Vanillin (compound 5), 5-amino-tetrazole (compound 2) and acetyl benzyl acetate (compound 6). So far, two reaction conditions are adopted: (1) sulfamic acid as catalyst and ethanol as solvent under reflux condition; and (2) iodine as catalyst and isopropanol as solvent under reflux condition.

1.3. Materials

MPP⁺, MPTP (1-methyl-4-phenyl-1,2,3,6-tetrahydropyridine) and nomifensine were obtained from Sigma-Aldrich (Germany). Hoechst 33342 and were purchased from Molecular Probes (Eugene, Oreg., USA). MTT, phenylmethanesulfonyl fluoride (PMSF) and RIPA lysis buffer were purchased from Sigma-Aldrich (St. Louis, Mo., USA). H89 were purchased from Calbiochem (Cambridge, Mass., USA). Primary antibodies for PKA, phosphorylated PKA, CREB, phosphorylated CREB, Akt, phospho-Akt, beta-actin, and horseradish peroxidase-conjugated anti-rabbit were purchased from Cell Signaling (Danvers, Mass., USA). Anti-TH antibody was obtained from Milipore (USA). LDH kit and phosphatase inhibitor cocktail were purchased from Roche Applied Science (Indianapolis, Ind., USA). Alexa Fluor® 488 anti-mouse antibody, Gibco® fetal bovine serum (FBS), and penicillin-streptomycin (PS) were purchased from Life Technologies (Grand Island, N.Y., USA).

1.4. NIH Mice

NIH mice, sex in half, for acute toxicity assay was supplied by Guangzhou Medicinal Experiment Animal Center and the animal qualification code was 44007200007090 and the certificate number was SCXK (Guangdong) 2013-0002. The animals were kept in the SPF-grade animal laboratory which was conformed to the SPF grade requirement of animal testing facility, where temperature was within the range of 22° C. (±2° C.) and the humidity was in the range of 30-70%. The diurnal lighting and darkness cycle was 12 hours. The air change per hour was in the range of 10-20 times. The approval no. of the SPF animal laboratory was SYXK (Guangdong) 2005-0062. The rat chow was the SPF-grade full pellet for mouse, which was bought from Guangdong Medicinal Laboratory Animal Center. The nutritional values and the sanitation condition were conformed to the SPF-grade requirement for animal testing. Antiseptic water were given ad libitum.

1.5. Cell Culture

The human neuroblastoma SH-SY5Y cells were purchased from America Tissue Type Collection. The cells were maintained in DMEM medium supplemented with 10% fetal bovine serum and penicillin/streptomycin (100 U/mL; 100 μg/mL) in a 37° C., 5% CO2 incubator. All experiments were carried out 48 hours after the cells were seeded.

1.6. Acute Toxicity Assay

NIH mice were randomly divided into 6 groups. Ten NIH mice, sex in half, were assigned to for each group. The groups include 1) Intravenous injection for blank control: intravenous injection of saline; 2) Intravenous injection for solvent control: intravenous injection of blank solvent (15% HS 15); 3) Intravenous injection for drug: intravenous injection of the max dose of compound BHDPC (0.5 mg/ml, it is 5 mg/kg); 4) oral gavage for blank control: oral gavage of saline; 5) oral gavage for solvent control: oral gavage of blank solvent (15% HS 15; and 6); oral gavage for drug: oral gavage of the max dose of compound BHDPC (0.5 mg/ml, administrated again 4 hours later, it is 10 mg/kg). The method of administration consists of warming up all the solutions in 37° C. water bath for 15 min before administration and administration of the solutions at a volume of 0.2 ml/20 g for each mouse. All mice were observed 5 min, 15 min, 30 min, 1 h, 2 h, 4 h, 8 h after dosing, and continued for 14 days, to figure out their survival situation and physiological state (including mentality, hair, breathing, change of movement frequency and characteristics, cardiovascular indications, salivary secretion, eating, drinking and defecation condition).

1.7. MTT Assay

The percentage of surviving cells was estimated by determining the activity of mitochondrial dehydrogenases with 3(4,5-dimethylthiazol-2-yl)-2,5-diphenyltetrazolium bromide (MTT) assay. After drug treatment, cells were incubated at 37° C. for 4 h in 0.5 mg/mL MTT solution. The medium was then removed, and 100 μL of DMSO was added to each well to dissolve the violet formazan crystals. The absorbance of the samples was measured at a wavelength of 570 nm with 655 nm as a reference wavelength. All values were normalized to the control groups.

1.8. Lactate Dehydrogenase (LDH) Assay

Cell cytotoxicity was also determined by measuring the activity of LDH released into the incubation medium when cellular membranes were damaged. Cells were seeded at 96-well plates (5×10³ cells/well). After treatment, the released LDH activity in the medium was measured according to the instructions of the Cytotoxicity Detection Kit (Roche Applied Science, Mannheim, Germany). Absorbance at 490 nm was measured using SpectraMax M5. All values of LDH released were normalized to the control group.

1.9. Caspase 3 Activity Assay

After treatment, the activity of caspase 3 was measured using the commercially available EnzChek Caspase-3 Assay Kit (Invitrogen, USA) according to the manufacturer's protocol. Briefly, SH-SY5Y cells were lysed in lysis buffer and centrifuged at 12,500×g for 5 min. 15 μL of cell lysate was incubated with 50 μL of 2× substrate working solution at room temperature for 30 min in 96-well plates. The fluorescence intensity was then determined by a SpectraMax M5 microplate reader at an excitation wavelength of 490 nm and emission at 520 nm. The fluorescence intensity of each sample was normalized to the protein concentration of sample. All values of % caspase 3 activities were normalized to the control group.

1.10. Measurement of Mitochondrial Membrane Potential (Δφm)

JC-1 dye was used to monitor mitochondrial integrity. In brief, SH-SY5Y cells were seeded into black 96-well plates (5×10³ cells/well). After treatment, the cells were incubated with JC-1 (10 μg/mL in medium) at 37° C. for 15 min and then washed twice with PBS. For signal quantification, the intensity of red fluorescence (excitation 560 nm, emission 595 nm) and green fluorescence (excitation 485 nm, emission 535 nm) were determined using a SpectraMax M5. Mitochondrial membrane potential (Δφm) was calculated as the ratio of JC-1 red/green fluorescence intensity and the value was normalized to the control group.

1.11. Western Blotting

After treatment, SH-SY5Y cells were collected and washed three times with ice-cold PBS. Then the harvested cells were lysed on ice for 30 min in RIPA lysis buffer containing 1% PMSF and 1% Protease Inhibitor Cocktail and centrifuged at 12,500×g for 20 min at 4° C. The supernatant was collected and protein concentrations were determined using the BCA protein assay kit (Thermo Scientific Pierce). Aliquots of protein samples (30 μg) were boiled for 5 min at 95° C. and electrophoresed on SDS-PAGE (10% (w/v) polyacrylamide gel) and then transferred to a polyvinylidene difluoride (PVDF) membrane (Bio-Rad, Hercules, Calif.). Subsequently, the membrane was blocked with 5% (w/v) non-fat milk in PBST (PBS containing 0.1% Tween-20) for 2 h at room temperature. The blots were incubated overnight at 4° C. with primary antibodies. After washed with PBST for 20 min at room temperature, the membranes were further incubated with horseradish peroxidase-conjugated secondary antibodies for 2 h at room temperature. Finally, protein bands were visualized using an ECL plus Western blotting detection reagents (GE Healthcare, Piscataway, N.J., USA). The membranes were then scanned on a Bio-Rad ChemiDoc XRS Imaging System and the intensity of the protein bands were analyzed using the Bio-Rad Quantity One Software (4.5.2).

1.12. Primary Cerebellar Granule Neuron Cultures

Rat CGNs were prepared from 8-day-old Sprague-Dawley rats (The Animal Care Facility, The Hong Kong Polytechnic University) as described in our previous publication. Briefly, neurons were seeded at a density of 2.7×10⁵ cells/ml in basal modified Eagle's medium (Invitrogen) containing 10% fetal bovine serum, 25 mM KCl, 2 mM glutamine, and penicillin (100 units/ml)/streptomycin (100 μg/ml). Cytosine arabinoside (10 μM) was added to the culture medium 24 h after plating to limit the growth of non-neuronal cells. With the use of this protocol, more than 95% of the cultured cells were granule neur

1.13. OPCs Culture

Purified OPC cultures were prepared as described. In brief, primary rat mixed glial cell cultures were isolated from whole brains of postnatal day (P) 2 rats, dissociated into single cells, and cultured into poly-D-lysine (PDL) coated T75 tissue culture flasks. Plating medium consisted of Dulbecco's modified Eagle's medium (DMEM, Invitrogen, Carlsbad, Calif.) supplemented with 10% fetal bovine serum (FBS; Invitrogen, Carlsbad, Calif.), 100 mM Mycozap. Tissue cultures were maintained at 37° C. in a humidified 5% CO2 incubator, and medium was exchanged every 3 days. Once confluent (after 10-15 days), microglia were separated by mechanical shaking of flasks on a rotary shaker for 60 mM at 250 rpm and removed. After addition of fresh medium, the remaining cells were allowed to recover overnight before repeating the mechanical shaking for an additional 16 h at 200 rpm to isolate OPCs. To ensure purity of OPC cultures, the isolated cells were transferred to a tissue culture dish, from which the loosely attached OPCs were detached by gentle shaking after 60 min, leaving behind attached microglia and astrocytes. OPCs were plated onto PDL coated 96 well plates using an automated dispenser and allowed to adhere to the plates over the next 1-2 days.

1.14. EdU Incorporation Assay

OPCs was incubated with BHDPC or 0.1% DMSO in the proliferation medium for two days were allowed to incorporate 5 uM EdU (Click-iT™ kit, Invitrogen™, OR, USA) for 4 h and fixed with 4% PFA for 15 min. Cells were washed again and incubated with 0.5% Triton X-100-based permeabilization buffer for 15 min. For the Click reaction, cells were incubated with Click-iT reaction buffer for 30 min and wash again with permeabilization buffer. All procedures were performed according to the manufacturer's instruction.

1.15. Immunostaining

OPC was incubated with BHDPC or 0.1% DMSO in the proliferation medium for two days. And then cells were fixed with 4% paraformaldehyde (PFA) for 10 min at room temperature, permeabilized and blocking with 0.3% Triton X-100, incubated with primary antibodies for overnight at 4° C. The final detection was made by incubating cells with FITC (488) or TRITC (594)-conjugated anti-rabbit or mouse IgG antibodies and counterstained with Hoechst33342. Photographs were captured by fluorescence microscope.

1.16. Cerebellar Slice Cultures

Following decapitation, brains of post-natal Day 9-10 SD rat were dissected out and sagittal slices (300 μm) of the cerebellum were immediately cut using a McIlwain tissue chopper, the slices were then isolated in Eagle's medium with Earle's salts medium (MEM) on ice and then placed on Millipore Millicell-CM™ organotypic culture inserts in pre-warmed medium containing MEM, Earle's balanced salt solution, heat-inactivated horse serum, GlutaMAX™, Fungizone®, and penicillin-streptomycin (each from Invitrogen), and glucose (Sigma). Compounds were added at the desired concentrations after 1 day in culture, and fresh medium supplemented with compounds every 2 days. Neuronal death was induced by adding MPP⁺ at 10 μM or 30 μM from day 2 in culture, BHDPC was added at 10 μM or 30 μM together with MPP⁺.

After 3-4 days treatment of MPP⁺ together with BHDPC, slices were fixed with 4% paraformaldehyde and then stained by immunohistochemistry. For the observation of neuronal protection effect by BHDPC in MPP⁺ toxicity, PI was added into the medium 20 minutes before the fixation and a quick observation and photographed was performed by Zeiss Observer.A1 fluorescent microscope and AxioVision digital image processing software. Three to four separate slice isolations (about 4-5 pups/isolation) were used, with 3-5 slices analysed from each isolation for each factor and dose.

1.17. Cerebellar Slice Culture Immunohistochemistry

Slices were fixed with 4% paraformaldehyde at room temperature for 1 hour, blocked with 5% donkey serum, 0.3% Triton™ X-100, and then incubated in primary antibodies for 2 days at 4° C. After washing in PBS, the sections were incubated at room temperature for 3-4 hours with fluorophore conjugated secondary antibodies (Life Technology) against the immunoglobulin of the species from which the primary antibody was generated. Upon completion of immunostaining, sections were briefly stained with Hoechst 33342 to reveal the cell nuclei, and then mounted with FluorSave™ Reagent (Merk 345789). Confocal z-stacks were acquired (at 1.3 μm intervals and 10-15 images were acquired per stack) with a Leica SPE confocal microscope and images analysed using NIH ImageJ. Only slices or area with intact cytoarchitecture were chosen for analysis. The density of PI stained nuclei in granular layer (NeuN staining) was applied for detecting neonatal damage/death.

1.18. Zebrafish Maintenance and Collection of Eggs

The AB strain of wild type zebrafish (Danio rerio) was maintained as described in the Zebrafish Handbook. Zebrafish were staged by days post fertilization (pdf). For breeding, groups of male and female (3:2) adult zebrafish were placed in 10-L plastic aquarium equipped with spawning nets in the evening. In the following morning, eggs were collected from the breeding group tanks. Normally developed fertilized eggs were selected under a stereomicroscope for further studies. The MPTP manipulation was performed with appropriate safety precautions and all MPTP-containing water was bleached.

1.19. Whole-Mount Immunostaining with Antibody Against Tyrosine Hydroxylase

Whole-mount immunostaining in zebrafish was performed as previously described. 3 dpf Zebrafish larvae were fixed with 4% paraformaldehyde in PBS for 30 min, Tyrosine hydrogenase (TH) staining was performed as previously described. Briefly, zebrafish were fixed in 4% paraformaldehyde in PBS for 5 h. Fixed samples were blocked (2% lamb serum and 0.1% BSA in PBST) for 1 h at room temperature. A mouse monoclonal anti-tyrosine hydroxylase (TH) antibody (Millipore, Billerica, MD, USA) was used as the primary antibody and incubated with samples overnight at 4° C. On the next day, samples were washed six times with PBST (each wash lasted 30 min), followed by incubation with Alexa Fluor® 488 goat anti-mouse antibodies. After immunostaining, zebrafish were mounted with 3.5% methylcellulose and photographed. Semi-quantification of the area of TH⁺ cells was assessed by an investigator, unaware of the drug treatment, using Image-J software. Results are expressed as percentage of area of TH⁺ cells in untreated normal control groups.

1.20. Statistical Analysis

Statistical analysis was performed using the GraphPad Prism statistical software (GraphPad software, Inc., San Diego, Calif.). All experiments were performed in triplicate. Data are expressed as means±standard deviation (SD). Statistical analysis was done by one-way ANOVA followed with Tukey's multiple comparison, with p<0.05 considered as statistically significance.

### Example 2

### Test of Cytotoxicity of BHDPC in SH-SY5Y Cells and NIH Mice

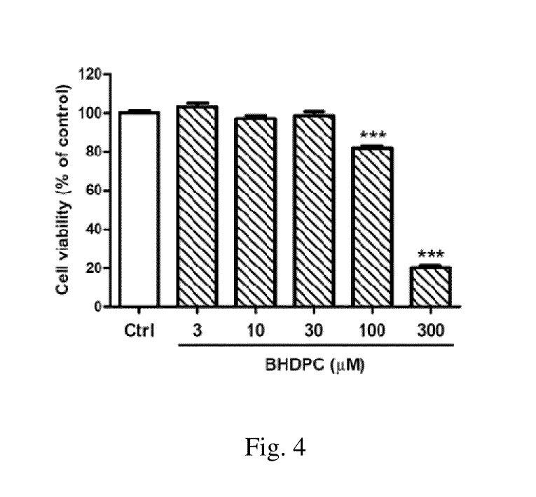

To evaluate the cytotoxicity of BHDPC, SH-SY5Y cells were incubated with various concentrations of BHDPC for 24 h and the cell viability was determined using MTT assay. As shown in FIG. 4, BHDPC at 3 to 30 μM did not cause any cytotoxicity and was used for further study.

To evaluate the acute toxicity of BHDPC in vivo, NIH mice were administrated with the maximum dose of BHDPC, to observe whether compound BHDPC is toxicity to mice. Intravenous injection and oral gavage are investigated separately. Results show that NIH mice, sex in half, after administrated with the maximum dose (5 mg/kg, iv and 10 mg/kg, oral), no toxic effects were observed in either intravenous injection group or oral gavage group, and all mice survived. This suggests that BHDPC has no toxicity to NIH mice at the dose of 5 mg/kg, iv and 10 mg/kg, oral.

### Example 3

### Neuroprotective Effect of BHDPC on SH-SY5Y Cells

1-Methyl-4-phenylpyridinium ion (MPP⁺), a Parkinsonism-inducing neurotoxin, is a metabolite of 1-methyl-4-phenyl-1,2,3,6-tetrahydropyridine (MPTP) catalyzed by the enzyme MAO-B in the brain. It can be taken up by dopaminergic neurons via dopamine transporter and further cause damage to dopaminergic neurons in the substantia nigra. Consequently, the MPP⁺/MPTP model is an excellent tool for the study of axonal degeneration and screening potential neuroprotective drugs for treatment, prevention or restoration of axonal pathology in such as Parkinson's disease.

3.1 BHDPC Reduced MPP⁺-Induced Cytotoxicity in Neuroblastoma SH-SY5Y Cells

To test the neuroprotective effects of BHDPC, SH-SY5Y cells were pre-treated with gradually increasing concentrations of BHDPC for 2 h and then treated with 1 mM MPP⁺ for 24 hours. Cell viability was measured using the MTT assay. As shown in FIG. SA, BHDPC prevented MPP⁺-induced dopaminergic neuronal death at 30 μM in SH-SY5Y cells.

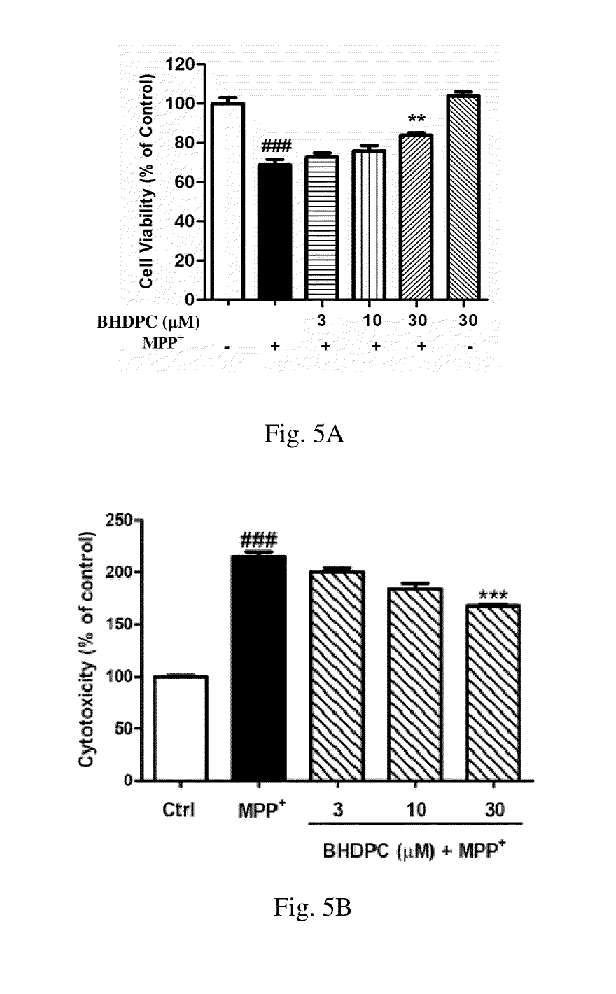

The protective activity of BHDPC was also confirmed by the lactate dehydrogenase (LDH) assay. SH-SY5Y cells were treated with BHDPC for 2 h before exposed to MPP⁺ for 36 h. As shown in FIG. 5B, pre-treatment with 3, 10 and 30 μM of BHDPC for 2 h markedly reduced MPP⁺-induced LDH leakage in a dose-dependent manner, from 209% to 200%, 184% and 170%, respectively.

3.2 BHDPC Decreased MPP⁺-Induced Caspase 3 Activation and Mitochondrial Membrane Potential Loss

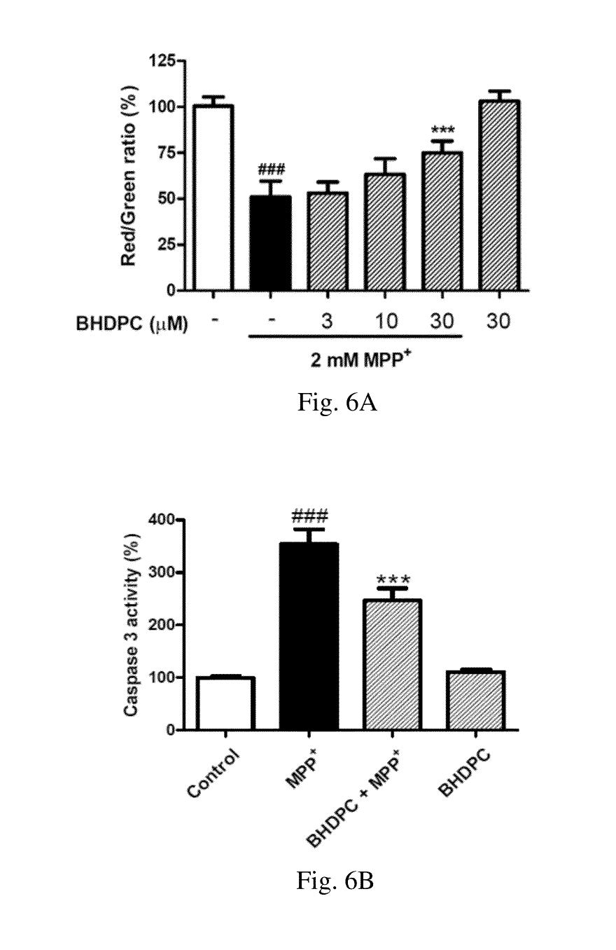

Loss of mitochondrial membrane potential and apoptotic nuclei were hallmarks for early and late stage of apoptosis. To determine whether BHDPC could reduce MPP⁺-induced mitochondrial membrane potential (Δφm) loss, the Δφm in SH-SY5Y cells was assessed by analyzing the red/green fluorescent intensity ratio of JC-1 staining (FIG. 6A). Exposure of SH-SY5Y cells to 2 mM MPP⁺ resulted in an increase in green fluorescence intensity indicating Δφm dissipation Pre-treatment with BHDPC at 3, 10 and 30 μM for 2 h attenuated MPP⁺-induced Δφm loss in a concentration-dependent manner, from 51% to 53%, 64% and 75%, respectively compared to the control group.

Caspase 3 activation plays a key role in the execution-phase of the apoptosis. As shown in FIG. 6B, treatment of cells with 2 mM MPP⁺ for 36 h increased caspase 3 activity by more than 3.5-fold relative to the control group. In contrast, pre-treatment with BHDPC at 30 μM significantly reduced MPP⁺-induced caspase 3 activation. BHDPC alone did not affect caspase 3 activity.

3.3 PKA Activation Involved in the Protective Effect of BHDPC

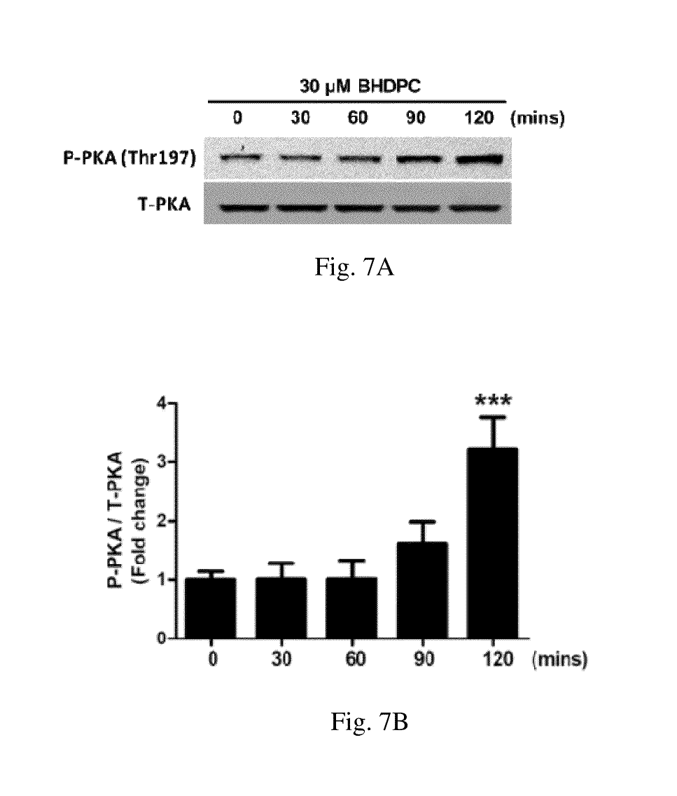

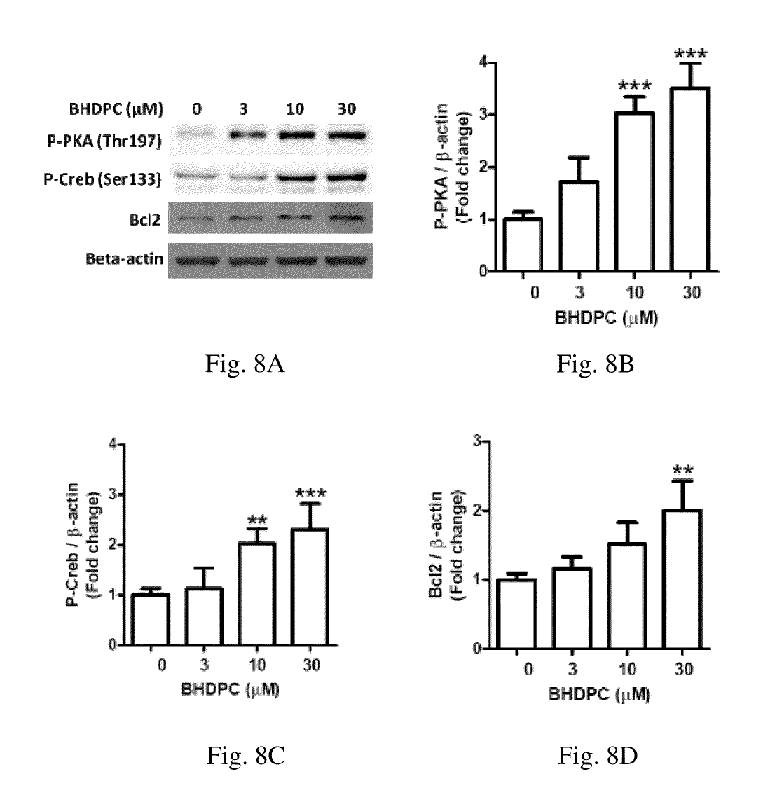

To determine which survival signaling pathway is regulated by BHDPC, SH-SY5Y cells were pre-treated with 30 μM BHDPC for 2 h, and then the phosphorylation of PKA at 0, 30, 60, 90, 120 mM were examined by Western blot analysis. As shown in FIGS. 7A and 7B, BHDPC gradually increased the phosphorylation intensity of PKA in SH-SY5Y cells; particularly at 90 and 120 min. The inventors also determined whether BHDPC affected the phosphorylation of CREB. The phosphorylation intensities of PKA and CREB were also increased by BHDPC in a dose-dependent manner (FIGS. 8A, 8B and 8C). Bcl2 is a well-known anti-apoptotic protein. The inventors determined whether BHDPC affected the expression of Bcl2. After treatment with different concentration of BHDPC, the expression of Bcl2 was up-regulated at dose-dependent manner (FIGS. 8A and 8D), suggesting that PKA/CREB signaling pathway could be induced by BHDPC.

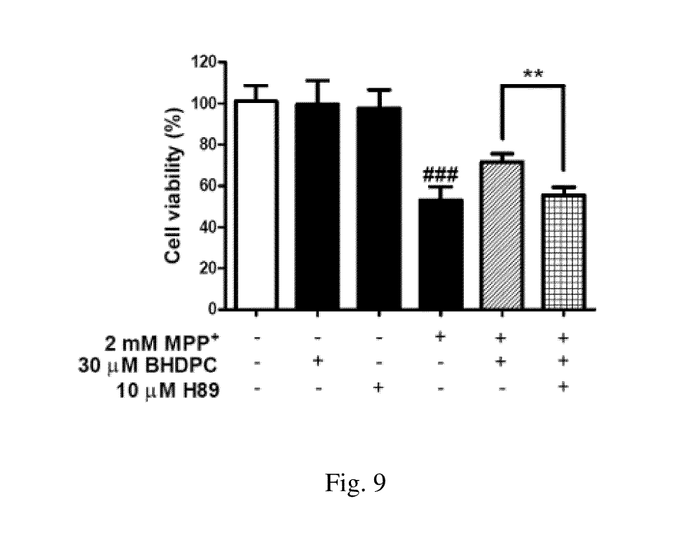

To further confirm the involvement of PKA activation in the protective effects of BHDPC, H89, a PKA inhibitor was used to measure cell survival under MPP⁺ cytotoxicity (FIG. 9). The results indicated that the neuroprotective effect of BHDPC was abolished by H89, suggesting that PKA activation is involved in the neuroprotective effects of BHDPC.

### Example 4

### Neuroprotective Effect of BHDPC on Oligodendroglial Progenitor Cells (OPCs)

4.1 BHDPC Promoted the Proliferation of OPCs

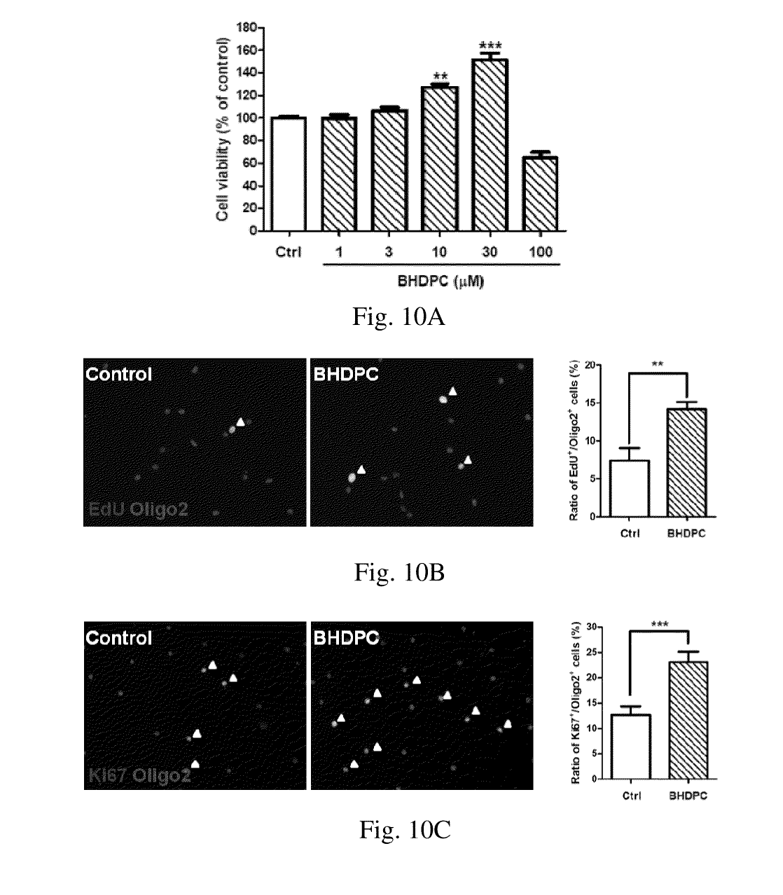

To test the effect of BHDPC on primary OPC culture, OPCs were treated with BHDPC for 48 h and the cell viability was determined using MTT assay. The results showed that BHDPC markedly promoted OPCs proliferation in a concentration-dependent manner (FIG. 10A). Compared with the control group, 1, 3, 10 and 30 μM BHDPC increased the viability to 100%, 106%, 127% and 152%, respectively, although some cytotoxicity was observed at 100 μM BHDPC (65%). The proliferation activity of BHDPC was also confirmed by the EdU incorporative assay. As shown in FIG. 10B, the ratio of EdU+/oligo2 cells was increased by treatment with 30 μM BHDPC. The immunostaining also revealed that BHDPC increased the ratio of Ki67+/oligo2 cells (FIG. 10C). It suggests that BHDPC was able to promote the proliferation of OPCs.

4.2 BHDPC Suppressed Hydrogen Peroxide and LPS-Induced OPC Death

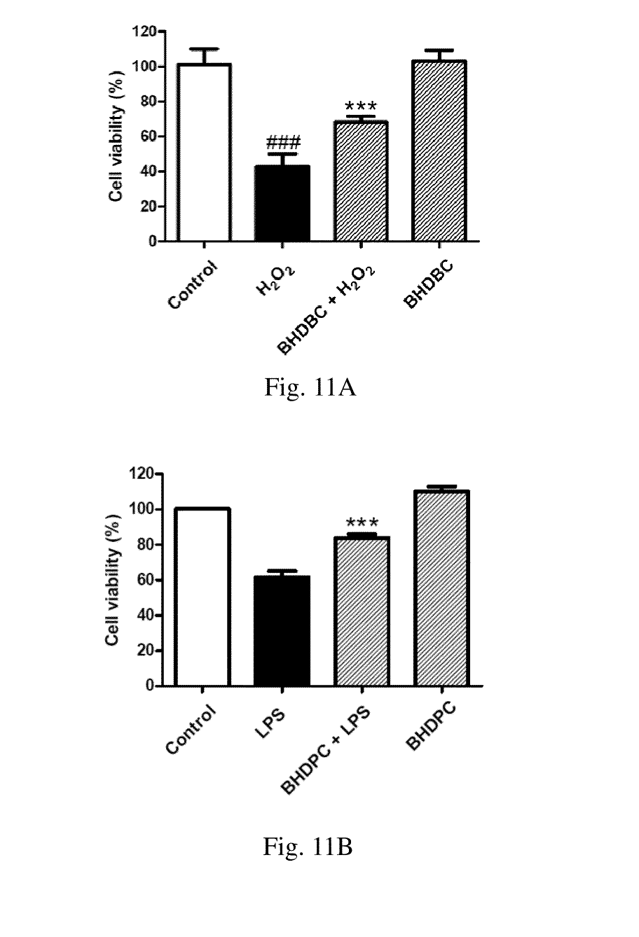

OPCs were treated with 30 μM BHDPC with or without 200 μM hydrogen peroxide for 6 h. As shown in FIG. 11A, hydrogen peroxide caused dominant cell death (44%), whereas pre-treatment with BHDPC dramatically attenuated the hydrogen peroxide-induced cell death.

Previous study reported that the LPS/IFNg inflammatory stimuli induced cytotoxicity in OPCs. To further exam the protective effects of BHDPC on OPCs, OPCs were pretreatment with 30 M BHDPC for 2 h and exposed to LPS for another 24 h. The result of MTT assay in FIG. 11B showed that LPS significantly decrease the cell viability which was observed comparatively to untreated cells, whereas BHDPC reduced LPS-induced cell death. These data with oxidative stress and inflammation-induced cell death suggest that BHDPC provided the pro-survival effects to OPCs.

### Example 5

### Neuroprotective Effect of BHDPC on CGNs

5.1 BHDPC Attenuated MPP⁺-Induced Neurotoxicity on Primary Cerebellar Granule Neurons (CGNs)

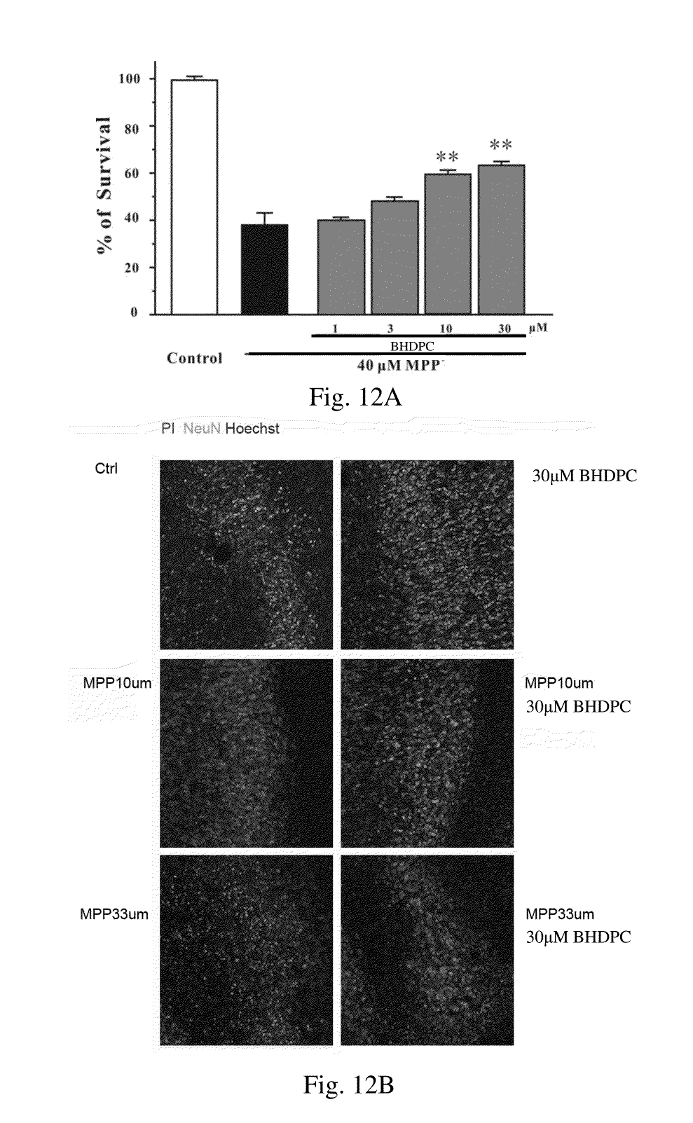

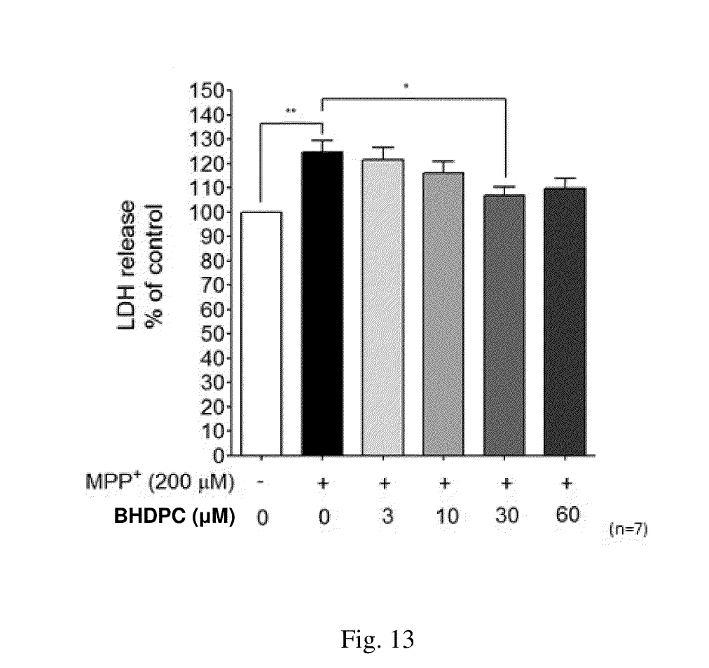

To further confirm the effect of BHDPC on primary neurons, mice cerebellar granule neurons were treated with BHDPC at the indicated concentrations for 2 hours and then exposed to 40 μM MPP⁺. Cell viability was measured by the MTT assay at 24 hours after the MPP⁺ challenge. As shown in FIG. 12A, BHDPC reduced MPP⁺-induced dopaminergic neuronal death at 10 and 30 μM in cerebellar granule cells.

5.2 BHDPC Enhanced the Viability of Cerebellar Neuronal Cells Exposed to MPP⁺ in Cerebellar Tissue Slices Culture

Cerebellar tissue slices culture is the co-culture of different CNS cells, is good model to mimic the in vivo condition of brain tissue. As shown in FIG. 12B, the brain slices were exposed to MPP⁺ for 3 days, which caused severe tissue damage. Propidium iodide (PI) staining in lived cells showed exposure of 33 μM MPP⁺ in such co-culture resulted in an increase in CNS cells death. The immunostaining revealed that the number of NeuN positive neuronal cells was significantly reduced in MPP⁺-treated brain slice. However, treatment of BHDPCs attenuated the MPP⁺-induced CNS cells death and neurons death in brain slices, was consistent with the results of the protective effects of BHDPC in OPCs and neurons.

### Example 6

### Neuroprotective Effect and Neural Regeneration Potential of BHDPC in Primary Cortical Neurons

6.1 Neuroprotective Effect of BHDPC in Primary Cortical Neuron

As shown in FIG. 13, primary cortical neuron cells were treated with BHDPC at the indicated concentrations for 2 hours and then exposed to 200 μM MPP⁺. LDH release was measured at 24 hours after the MPP⁺ challenge. The result showed that BHDPC prevented MPP⁺-induced neuronal death in primary cortical neurons.

6.2 Neural Regeneration Potential of BHDPC in Primary Cortical Neuron

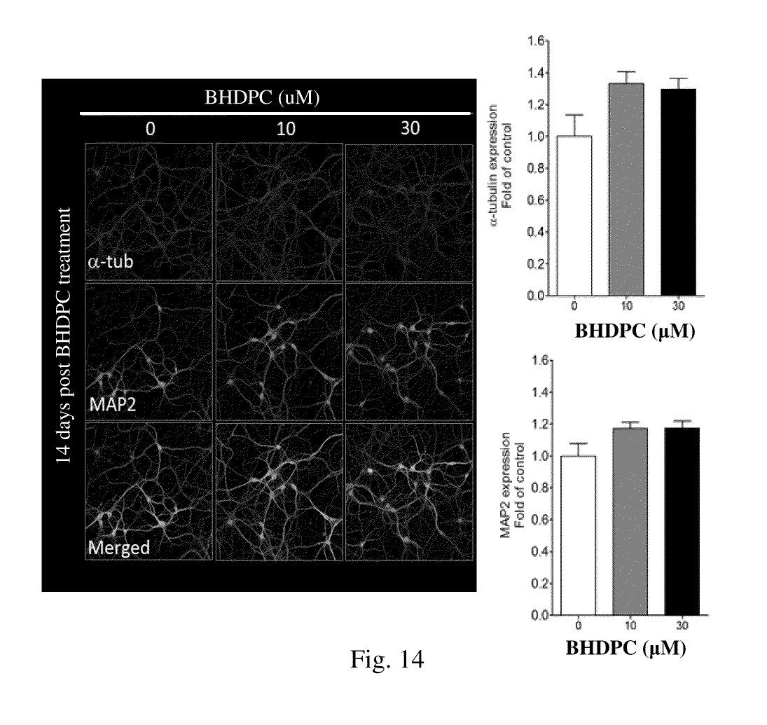

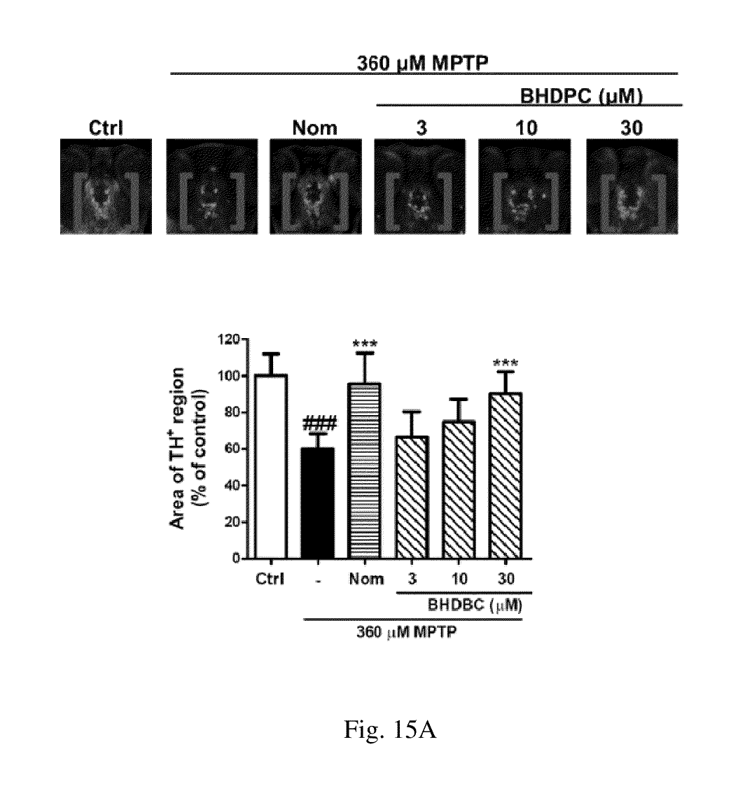

As shown in FIG. 14, the treatment of primary cortical neuron cell culture with BHDPC for 14 days induced increase levels of the cytoskeletal proteins, microtubule-associated protein (MAP2), and α-tubulin (a-tub), suggesting that BHDPC has neurite outgrowth promoting effect.

### Example 7

### BHDPC Suppressed MPTP-Induced Dopaminergic Neurons Loss of Zebrafish

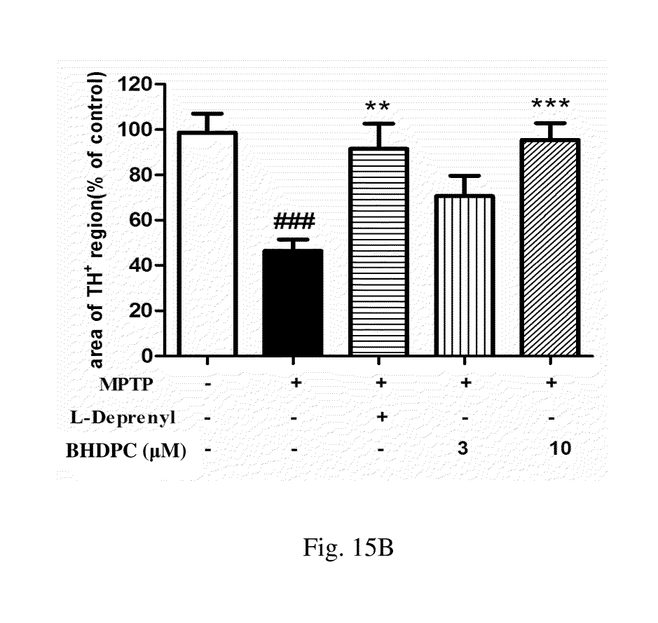

The in vitro study demonstrated a neuroprotective effect of BHDPC on MPP⁺-induced neuronal cells death. In this example, the in vivo animal model was used to determine the neuroprotective effect of BHDPC. Anti-tyrosine hydroxylase (TH) whole mount immunostaining was used to determine dopaminergic neuronal populations in zebrafish larvae (FIG. 15A). TH activity is the key enzyme responsible for dopamine biosynthesis in the CNS. The exposure of 1 dpf zebrafish embryos to 360 μM MPTP for 48 h dramatically resulted in TH+ density reduction (60%) in the ventral diencephalic clusters compared with the untreated control group. The dopamine reuptake inhibitor nomifensine (Nom), which protected against MPTP-induced neurotoxicity in vivo was used as a positive control. The treatment of larvae with 30 μM of nomifensine, a positive control drug attenuated MPTP-induced neurotoxicity, with TH⁺ density reduced by 30% compared to the MPTP group. The treatment with 3, 10 and 30 μM of BHDPC could rescue dramatically TH⁺ density decrease almost to the normal level in a dose-dependent manner (66%, 74% and 90%, respectively). No toxicity was observed in the vital organs of the BHDPC treated animals compared to the control groups.

As shown in FIG. 15B, BHDPC protected against MPTP-induced DA neuron loss in zebrafish. L-deprenyl, which is a substituted phenethylamine used in treatment of early Parkinson's disease, was used as a positive control. The treatment of larvae with L-deprenyl attenuated MPTP-induced neurotoxicity, with significant increase in TH⁺ density as compared to the MPTP group. In addition, the treatment with 3 and 10 μM of BHDPC could rescue dramatically TH⁺ density decrease almost to the normal level in a dose-dependent manner.

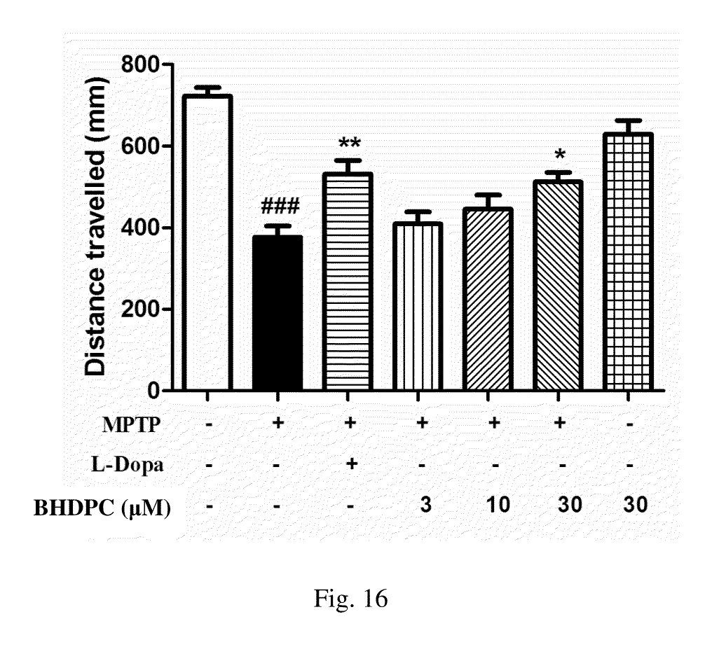

In addition, MPTP markedly altered the swimming behavior of the zebrafish as a consequence of DA neuronal injury. As shown in FIG. 16, the total distance travelled by the zebrafish larvae decreased significantly after exposure to MPTP. BHDPC ameliorated MPTP-induced deficit of swimming behavior. At the same condition, MPTP-induced deficit of swimming behavior were rescued by positive controls, levodopa (L-dopa) (FIG. 16). BHDPC treatment alone notably altered the swimming behaviour of normal zebrafish larvae (FIG. 16).

### Example 8

### Interaction of BHDPC with ROCK Enzymes

8.1 Enzyme Activity and Docking

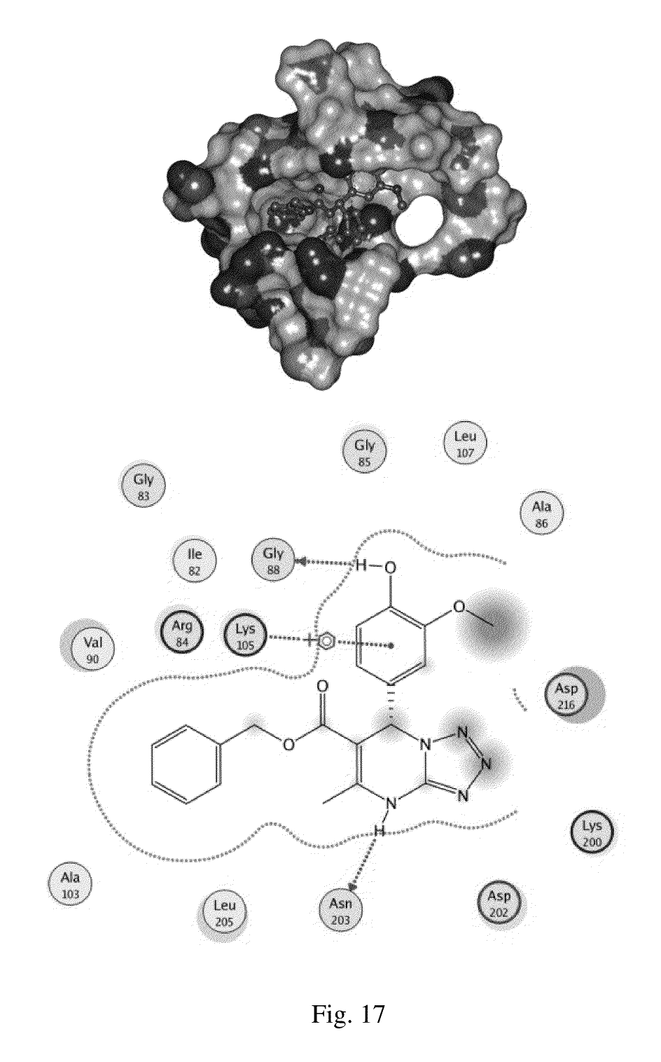

ROCK has two isoforms (ROCK1 and ROCK2) sharing the same downstream proteins. Bioassay and molecular docking are used to compare the characteristics of BHDPC on ROCK. Kinase activity assay showed that the IC50 value of BHDPC against ROCK1 was 13.7 μM whereas that against ROCK2 was 408.3 μM (Table 1). These indicated BHDPC exhibited better affinity for ROCK1 than ROCK2. Through molecular docking and molecular dynamics simulation, the interaction between the inhibitor and ROCK1 was shown in FIG. 17. Predicted result indicated that inhibitors and molecular recognition between ROCK1 mainly through van der Waals and hydrogen bonding interactions. The hydroxyl and amino groups of BHDPC formed two stable hydrogen bonds with Gly88 and Asn203 of ATP binding site; two benzene rings could produce strong van der Waals interactions with a plurality of hydrophobic residues, including Leu107, Ile82, Va190 and Leu205; Furthermore, positive ions of Lys and benzene of BHDPC formed cation-π interactions.

TABLE 1
Characteristics of BHDPC for ROCK inhibition

| | ROCK1 | ROCK 2 |
|---|---|---|
| | IC₅₀ (μM) | IC₅₀ (μM) |
| BHDPC | 13.7 | 408.3 |

Discussions

OPCs are a population of CNS cells that are distinct from neurons, oligodendrocytes, astrocytes and microglia. OPCs have been considered as first CNS cells to response to brain injury; they are highly sensitive to microenvironment changes which regulate their bio-processes such as survival, migration, proliferation, differentiation and cell fate. OPCs mature to oligodendrocytes, are necessary for axon integrity under physiological conditions. Oligodendrocytes dysfunction leads to axonal degeneration, a hallmark of neurodegeneration affect the normal function of neurons. In addition, recent studies showed that OPCs could give rise to neurons in vitro and in perinatal cerebral cortex and piriform cortex in vivo. Therefore, survival of OPCs is a critical factor to maintain the normal function of neuronal axon and neurons survival. The inventor has identified the new neuroprotectant named BHDPC is able to enhance OPCs proliferation and survival, illustrating that it may provide survival signals to OPCs cells to enhance the supportive role of OPCs in axon integrity and neurons survival.

Morphologic defects and functional change of mitochondria are showed in patients with neurodegenerative disorders, pointing toward the critical role of mitochondria defect in the cause of neurodegeneration. MPP⁺, an active metabolite of 1-Methyl-4-phenyl-1,2,3,6-tetrahydropyridine (MPTP), is a neurotoxin widely used to produce Parkinsonism. MPP⁺ is converted from MPTP by MAO-B of glial cells and further inhibit mitochondrial complex I of the electron transport chain in neurons. The mitochondria dysfunction by MPP⁺ causes ATP depletion and stimulates the generation of reactive oxygen species (ROS) to cause neuronal cells death. In above experiments, treatment with BHDBC alone could normalize the MPP⁺-induced Δφm loss and mitochondrial depolarization, providing mechanistic evidence to support that BHDPC prevents neuronal mitochondria from MPP⁺ neurotoxicity.

Neurodegenerative diseases exhibit complex features of apoptotic neuronal death, regulated by the apoptosis-related proteins. It has been reported that strategies to mediate apoptotic-related proteins may be potential therapeutics. MPP⁺ reacts with mitochondria complex I and leads to cause damage to the mitochondrial membrane and results in the collapse of the Δφm, irreversible oxidative damage and activation of the apoptotic cascade. Apoptotic marker, Δφm caspase 3, and LDH are affected by the MPP⁺-mediated mitochondrial apoptotic pathway. BHDPC which exhibited effective neuroprotective effects against MPP⁺-induced neurotoxicity in SH-SY5Y cells and primary CGNs and primary cortical neuron. It has also been found that a decrease in Δφm, activation of caspase 3 and LDH release induced by MPP⁺ could be restored by the anti-apoptotic effects of BHDPC.

Neurotoxin-induced PD models of zebrafish have been successfully used to identify numerous neuroprotectants. The catecholaminergic neurotoxicity of MPTP has been shown to dominate the DA neuronal death and leads to locomotion behavior deficiency, thus, has been demonstrated to be an appropriate model for PD. In the above examples, TH immunostaining of zebrafish showed that the immunopositive area of DA neuron had been significantly reduced by MPTP; whereas the loss of DA neuron could be effectively attenuated by BHDPC. Moreover, MPTP-induced deficit of swimming behavior in zebrafish were rescued by BHDPC. These provide confirmatory evidences supporting the observed neuroprotective effect of BHDPC in vitro.

PKA is a cyclic AMP (cAMP)-dependent protein kinase involved in the regulation of glycogen, sugar, and lipid metabolism. In neuronal cells, the PKA signaling pathway promotes cell survival and suppresses apoptosis by phosphorylation and inhibition of several downstream substrates. PKA first directly activates CREB, which binds the cAMP response element and further mediate the expression of downstream genes such as Bcl2, can confer to the stabilization of mitochondria. Induction of bcl-2 expression by phosphorylated CREB proteins during B-cell activation and rescue from apoptosis. The observed gradual increase in active PKA, active CREB and expression of Bcl2 following treatment with BHDPC indicates that BHDPC-regulated protective effects in SH-SY5Y cells are partly via PKA/CREB pathway.

In summary, it demonstrates that BHDPC not only reduces MPP⁺-induced SH-SY5Y cells and primary cortical neurons death but also significantly provides the pro-survival and proliferative effects to OPCs. These effect further support the coordinative neuroprotective effects of BHDBC against MPP⁺ on brain tissue slices and suppress MPTP-induced dopaminergic neurons loss of zebrafish. The mechanism of BHDPC in neurons is through the regulation of multiple pathways including (1) mediating Δφm/caspase 3 dependent apoptosis pathways; (2) activating PKA/CREB/Bcl2 signaling. In addition, the pro-survival and proliferative potential of BHDPC may confer to brain microenvironment to support neurons survival. The results provide the support for the development of BHDPC in treatment of PD and AD or other neurodegenerative diseases, particularly those associated with OPCs loss, such as multiple sclerosis.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

The pharmaceutically acceptable salt is selected from the group consisting of hydrochlorid, phosphate, sulphate, acetate, maleate, citrate, benzene sulfonate, toluenesulfonate, fumarate and tartrate.

What is claimed is:

1. A method of treating and/or preventing neurodegenerative disease comprising administrating a therapeutically effective amount of a compound of formula I or a pharmaceutically acceptable salt thereof to a subject in need thereof 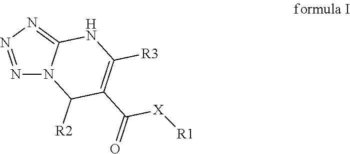
wherein R1, R2 and R3 are independently selected from the group consisting of H, unsubstituted or substituted (C1-C6) alkyl, (C1-C4) alkoxy, (C6-C12) aryloxy, and unsubstituted or substituted aryl; and
X is C, O or N.

2. The method of claim 1, wherein R1, R1, R2 and R3 are independently selected from the group consisting of H, unsubstituted or substituted (C1-C6) alkyl, (C1-C4) alkoxy, (C6-C12) aryloxy, and unsubstituted or substituted (C6-C12) aryl.

3. The method of claim 1, wherein R3 is halo or halo substituted straight or branched (C1-C6) alkyl.

4. The method of claim 1, wherein R1 and R2 are independently selected from the group consisting of five or six-membered N-heterocycle and benzoheterocycle.

5. The method of claim 1, wherein R1, R2 and R3 are independently (C1-C4) alkyl.

6. The method of claim 1, wherein R1, R2 and R3 are independently selected from the group consisting of phenylmethoxyl, phenylethoxyl or phenylpropoxyl.

7. The method of claim 1, wherein R1 and R2 are independently selected from the group consisting of phenyl, chlorobenzene, phenol or aniline.

8. The method of claim 1, wherein the compound is selected from the group consisting of benzyl 7-(4-hydroxy-3-methoxyphenyl)-5-methyl-4,7-dihydrotetrazolo[1,5-a]pyrimidine-6-carboxylate, 7-(4-hydroxy-3-methoxylphenyl)-5-methyl-6-carboxylic acid benzyl ester-4,7-dihydrotetrazolo[1,5-α]pyrimidine; 7-(3,4-diethoxylphenyl)-6-[N-(2-methoxylphenyl)methanamide]-5-methyl-4,7-dihydrotetrazolo[1,5-α]pyrimidine; 7-(4-phenylmethoxyl-3-methoxylphenyl)-6-(N-phenyl methanamide)-5-methyl-4,7-dihydrotetrazolo[1,5-α]pyrimidine; 7-(2-N-pyrimidine)-5-fluor-6-carboxylic acid benzyl ester-4,7-dihydrotetrazolo[1,5-a]pyrimidine; 7-phenyl-6-(N-phenylmethyl)methanamide-5-methyl-4,7-dihydrotetrazolo[1,5-α]pyrimidine; 7-(1,3-benzodioxol-pentene)-6-[N-(2-methoxylphenyl)methanamide]-5-methyl-4,7-dihydrotetrazolo[1,5-α]pyrimidine; 7-(3-methoxy-3′-nitro-4-hydroxylphenyl)-6-[N-(2-methoxylphenyl)methanamide]-5-methyl-4,7-dihydrotetrazolo[1,5-α]pyrimidine; 7-(4-phenylmethoxylphenyl)-6-[N-(2-pyridine)methanamide]-5-methyl-4,7-dihydrotetrazolo[1,5-α]pyrimidine; and 7-(4-methoxylphenyl)-6-carboxylic acid isopropyl ester-5-methyl-4,7-dihydrotetrazolo[1,5-α]pyrimidine.

9. The method of claim 1, wherein the compound is benzyl 7-(4-hydroxy-3-methoxyphenyl)-5-methyl-4,7-dihydrotetrazolo[1,5-a]pyrimidine-6-carboxylate.

10. The method of claim 1, wherein the neurodegenerative disease is selected from the group consisting of Parkinson disease, Alzheimer disease, Huntington's disease, multiple sclerosis and amyotrophic lateral sclerosis.

11. The method of claim 1, wherein the compound is administered to the subject by oral administration or by intravenous injection.

12. The method of claim 1, wherein the pharmaceutically acceptable salt is selected from the group consisting of hydrochlorid, phosphate, sulphate, acetate, maleate, citrate, benzene sulfonate, toluenesulfonate, fumarate and tartrate.

13. A pharmaceutical composition for treating and/or preventing neurodegerative disease comprising a compound of claim 1 and a pharmaceutically acceptable carrier.

14. The pharmaceutical composition of claim 13, wherein the neurodegenerative disease is selected from the group consisting of Parkinson disease, Alzheimer disease, Huntington's disease, multiple sclerosis and amyotrophic lateral sclerosis.
